(19) United States
(12) Patent Application Publication
(10) Pub. No.: US 2022/0230298 A1
(43) Pub. Date: Jul. 21, 2022

(54) DETERMINATION SYSTEM FOR ACHIEVEMENT DEGREE OF PROCESS, MEASUREMENT DEVICE, AND INFORMATION PROCESSING DEVICE

(71) Applicant: CANON KABUSHIKI KAISHA, Tokyo (JP)

(72) Inventor: Toshifumi Kitamura, Shizuoka (JP)

(21) Appl. No.: 17/717,463

(22) Filed: Apr. 11, 2022

Related U.S. Application Data
(63) Continuation of application No. PCT/JP2020/034950, filed on Sep. 15, 2020.

(30) Foreign Application Priority Data
Oct. 23, 2019 (JP) 2019-193010

(51) Int. Cl. G06T 7/00; G06T 7/90; G06T 7/70

(52) U.S. Cl. CPC G06T 7/0002; G06T 2207/20092; G06T 7/70; G06T 7/90

(57) ABSTRACT

A determination system includes: a measurement unit configured to measure color information on a surface of an indicator having a discoloration area in which a color changes according to an achievement degree of a predetermined process; a determination unit configured to determine an initial defect in the indicator or the achievement degree of the predetermined process based on the color information of the discoloration area measured by the measurement unit; a storage unit configured to store a control parameter corresponding to a type of the indicator; and a processing unit configured to perform processing of setting the control parameter in the storage unit. The measurement unit is further configured to measure the color information of the discoloration area of the indicator based on the control parameter, stored in the storage unit, that corresponds to the type of the indicator to be measured.

### CROSS-REFERENCE TO RELATED APPLICATIONS

This application is a Continuation of International Patent Application No. PCT/JP2020/034950, filed Sep. 15, 2020, which claims the benefit of Japanese Patent Application No. 2019-193010, filed Oct. 23, 2019, both of which are hereby incorporated by reference herein in their entirety.

### BACKGROUND OF THE INVENTION

### Field of the Invention

The present invention relates to a determination system that determines an achievement degree of a process using an indicator, and to a measurement device and an information processing device of the determination system.

### Background Art

Sterilization processes are performed on sterilization targets such as medical instruments in a hospital. In order to determine an achievement degree of the sterilization process on the sterilization targets, so-called sterilization indicators such as a chemical indicator (hereinafter, CI) are used. A CI has a discoloration area that changes color in accordance with the achievement degree of the conditions required for the sterilization process, which uses a sterilizing agent (vapor, hydrogen peroxide, or the like). A common method of confirming the achievement degree of a sterilization process is to visually check the color change in the discoloration area of the CI. PTL 1 discloses a measurement device that measures the achievement degree of a sterilization process by optically reading the color of the discoloration area of a CI.

### CITATION LIST

### Patent Literature

PTL 1: Japanese Patent Laid-Open No. 2003-325646

Here, different CIs are used depending on the sterilization method used by a sterilizing device. A different sterilization method means, for example, a different sterilizing agent or a different sterilization process is used. The location of the discoloration area of the CI, the colors before and after the change, and so on may be different as well. In hospitals and the like, multiple sterilizing devices having different sterilization methods are used, and multiple types of CIs are used as a result. Because the way in which the color changes, the location, and the like of the discoloration area differs depending on the type of the CI, it is difficult for the measurement device disclosed in PTL 1 to handle new types of CIs newly introduced to the market.

### SUMMARY OF THE INVENTION

According to one aspect of the present invention, a determination system includes: a measurement unit configured to measure color information on a surface of an indicator having a discoloration area in which a color changes according to an achievement degree of a predetermined process; a determination unit configured to determine an initial defect in the indicator or the achievement degree of the predetermined process based on the color information of the discoloration area measured by the measurement unit; a storage unit configured to store a control parameter corresponding to a type of the indicator; and a processing unit configured to perform processing of setting the control parameter in the storage unit, wherein the measurement unit is further configured to measure the color information of the discoloration area of the indicator based on the control parameter, stored in the storage unit, that corresponds to the type of the indicator to be measured.

Further features of the present invention will become apparent from the following description of exemplary embodiments with reference to the attached drawings.

### BRIEF DESCRIPTION OF THE DRAWINGS

The accompanying drawings, which are incorporated in and constitute a part of the specification, illustrate embodiments of the invention and, together with the description, serve to explain principles of the invention.

FIG. 1 is a schematic diagram illustrating a determination system according to an embodiment.

FIG. 2A is a schematic diagram illustrating a spectrometer.

FIG. 2B is a schematic diagram illustrating a spectrometer.

FIG. 2C is a schematic diagram illustrating a spectrometer.

FIG. 3 is a function block diagram illustrating a determination system.

FIG. 4 is a diagram illustrating an example of a CI.

FIG. 5 is a diagram illustrating another example of a CI and a profile of the CI.

FIG. 6 is a diagram illustrating control parameters of the CI illustrated in FIG. 5.

FIG. 7 is a diagram illustrating yet another example of a CI and a profile of the CI.

FIG. 8 is a diagram illustrating control parameters of the CI illustrated in FIG. 7.

FIG. 9 is a flowchart illustrating location parameter setting processing according to an embodiment.

FIG. 10 is a diagram illustrating a display screen of a profile reading result according to an embodiment.

FIG. 11 is a flowchart illustrating color parameter setting processing according to an embodiment.

FIG. 12 is a flowchart illustrating control parameter obtainment processing according to an embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate.

Furthermore, in the attached drawings, the same reference numerals are given to the same or similar configurations, and redundant description thereof is omitted.

### First Embodiment

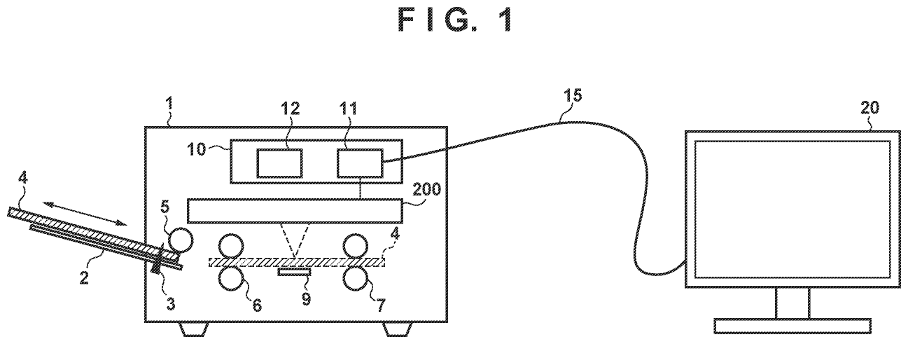

FIG. 1 is a schematic diagram of a determination system for the achievement degree of a sterilization process according to the present embodiment. The determination system includes a measurement device 1 for a CI 4, and a control device 20. The control device 20 can be constituted by a personal computer (“PC” hereinafter), a tablet, or the like, for example. The measurement device 1 and the control device 20 are communicably connected by a communication line 15. Although the present embodiment assumes that the measurement device 1 and the control device 20 communicate over the communication line 15, the measurement device 1 and the control device 20 can also be configured to communicate wirelessly. Furthermore, the measurement device 1 and the control device 20 can be configured to communicate over a network such as a wired LAN or a wireless LAN.

A sensor 3 of the measurement device 1 detects whether the CI 4 is placed on a tray 2. A roller 5 conveys the CI 4 into the measurement device 1. The CI 4 conveyed into the measurement device 1 is conveyed by an upstream roller 6 and a downstream roller 7 to a measurement region of a spectrometer 200. The spectrometer 200 measures information about the color of the surface of the CI 4 and outputs a measurement result to a control unit 10. The control unit 10 includes a CPU 11 and non-volatile memory 12. A white reference plate 9 for color correction of the spectrometer 200 is provided between the upstream roller 6 and the downstream roller 7. When the CI 4 is not between the spectrometer 200 and the white reference plate 9, the spectrometer 200 can measure information about the color of the surface of the white reference plate 9. After the measurement by the spectrometer 200, the CI 4 is discharged to the tray 2 by the upstream roller 6 and the downstream roller 7. Note that the measurement device 1 illustrated in FIG. 1 takes in and measures the CI 4 placed on the tray 2, and after measurement, discharges the CI 4 to the tray 2 again. However, a discharge tray may be provided on the opposite side of the tray 2 relative to the spectrometer 200, and the CI 4 may be discharged to the discharge tray after measurement.

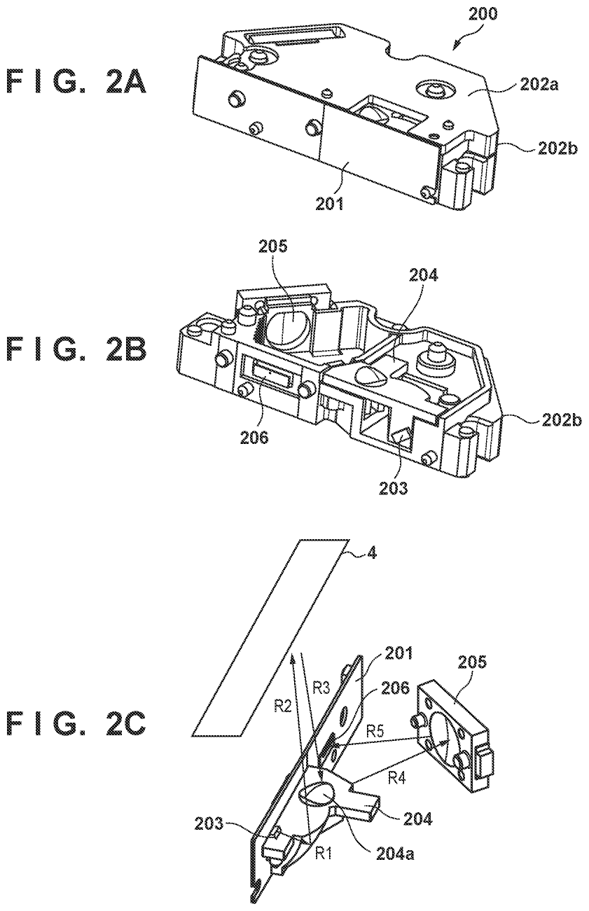

FIGS. 2A to 2C are schematic diagrams of the spectrometer 200. FIG. 2A is an exterior view of the spectrometer 200, and FIG. 2B illustrates a state in which a board 201 and a cover member 202a illustrated in FIG. 2A are removed from a housing 202b of the spectrometer 200. The board 201 is provided with a white LED 203, a line sensor 206, and circuitry for amplifying an output signal from the line sensor 206 and converting the output signal into a digital signal, and the like. Note that FIG. 2B illustrates the white LED 203 and the line sensor 206 as being located at the positions where the white LED 203 and line sensor 206 are located when the board 201 is attached to the housing 202b as illustrated in FIG. 2A. A light guide 204 is a light guiding member that integrates an illumination part, which guides light from the white LED 203 toward a measurement subject, with a focusing part, which focuses and guides reflected light from the measurement subject. The light emitted by the white LED 203 irradiates the measurement subject, i.e., the CI 4, via the light guide 204. The reflected light from the measurement subject is focused and guided by the focusing part of the light guide 204 and irradiates a diffraction grating 205. The diffraction grating 205 spectrally divides the reflected light according to the wavelength thereof. The line sensor 206 includes a plurality of light-receiving elements, each of which receives light in a predetermined wavelength range that is spectrally diffracted by the diffraction grating 205.

FIG. 2C is a descriptive diagram illustrating the path of light from when the light is emitted from the white LED 203 to when the light is incident on the line sensor 206. Light R1 emitted by the white LED 203 is reflected by a curved surface part of the light guide 204, and irradiates the CI 4 as irradiation light R2. Reflected light R3 from the CI 4 is incident on a light incidence part 204a of the light guide 204. The reflected light incident on the light incidence part 204a is focused and guided by the light guide 204, and irradiates the diffraction grating 205 as reflected light R4. Reflected light R5, which has been spectrally diffracted by the diffraction grating 205, is incident on the line sensor 206.

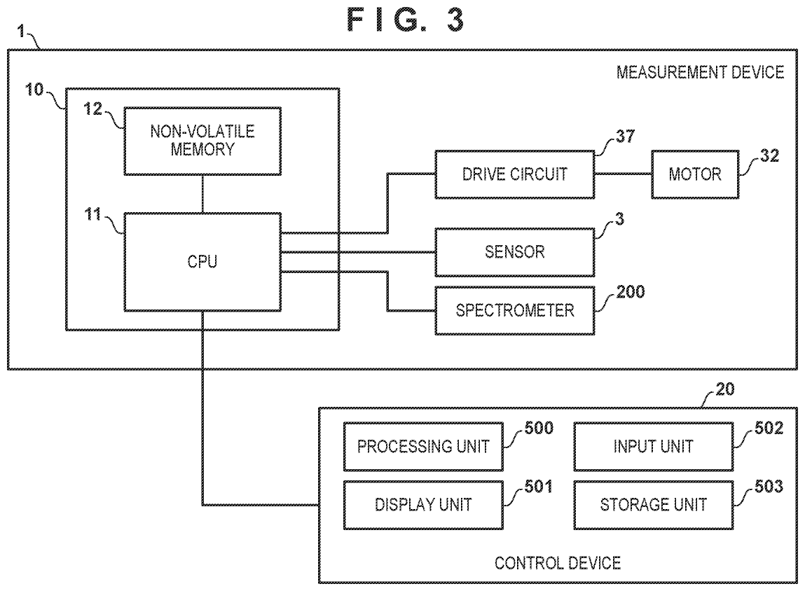

FIG. 3 is a function block diagram of the measurement device 1 and the control device 20. The CPU 11 controls the rotation of one or more motors 32 by controlling a drive circuit 37. The one or more motors 32 rotate the roller 5, the upstream roller 6, and the downstream roller 7. The CPU 11 also obtains a detection result from the sensor 3. Furthermore, the CPU 11 controls the measurement of the CI 4 by the spectrometer 200. Further still, the CPU 11 receives signals indicating the amount of light received from each light-receiving element in the line sensor 206 of the spectrometer 200, and obtains color information of the surface of the CI 4. The color information obtained by the CI 4 can be color information of any color space. The CPU 11 can determine the achievement degree of a sterilization process based on the color information of the discoloration area of the CI 4. The CPU 11 can also determine whether the CI 4 has an initial defect based on the color information of the discoloration area before the sterilization process. The CPU 11 sends a determination result to the control device 20.

The non-volatile memory 12 holds programs executed by the CPU 11, various types of information, and the like. The various types of information include control parameters used by the CPU 11 when measuring the CI 4, when determining the achievement degree of the sterilization process, when determining initial defects, and the like. The CPU 11 executes programs held in the non-volatile memory 12, and measures the CI 4 and the like using the various types of information held in the non-volatile memory 12. The configuration of the control device 20 will be described later.

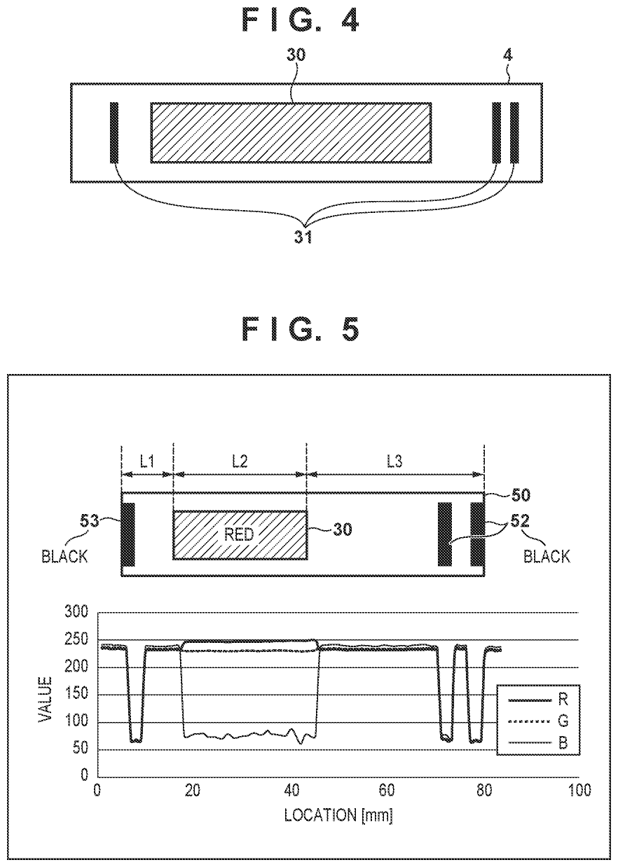

FIG. 4 illustrates an example of the CI 4. The CI 4 is a sheet-shaped test paper, and has on its surface a discoloration area 30 which has been subjected to a chemical process. The discoloration area 30 changes color in accordance with the achievement degree of the sterilization process. The CI 4 has a pattern 31. The pattern 31 is different depending on the type of the CI 4. As illustrated in FIG. 4, the pattern 31 is printed on the same surface of the CI 4 as the discoloration area 30, but at a different location than the discoloration area 30. The measurement device 1 is configured to convey the CI 4 in the long side direction of the CI 4. In the following descriptions, the long side direction of the CI is illustrated as the left-right direction in the figure, and in this case, the short side on the left side of the figure will be called a “first end”, and the short side on the right side of the figure will be called a “second end”. It is assumed that a user places the CI on the tray 2 such that the first end is a leading end when the CI placed on the tray 2 is convey into the measurement device 1.

FIG. 5 illustrates a CI 50 used in the descriptions of the present embodiment. The discoloration area 30 of the CI 50 is designed to be red before the sterilization process and change to yellow after the sterilization process. The pattern 31 of the CI 50 has one black line 53 at the first end and two black lines 52 at the second end. As illustrated in FIG. 5, the length of the discoloration area 30 in the long side direction is L2. The distance from the first end to the discoloration area 30 is L1, and the distance from the second end to the discoloration area 30 is L3. The lower part of the CI 50 in FIG. 5 indicates the profile of the CI 50 with respect to the color thereof before the sterilization process. The horizontal axis of the profile represents the position of the CI 50 in the long side direction, and the vertical axis represents the measured color information. Note that in this example, the color information is assumed to be color values in the RGB color space. In the present embodiment, the profile pertaining to the color of the CI (called simply a “profile” hereinafter) is the color information of the CI in a predetermined direction. Note that the predetermined direction is the direction parallel to the long side of the CI 4.

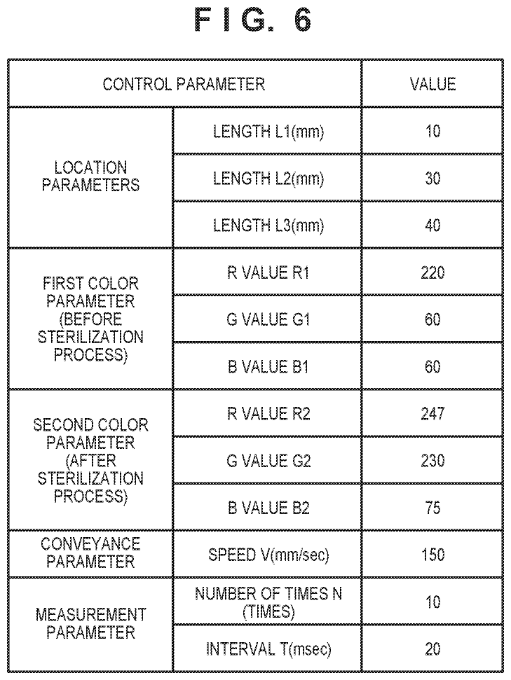

FIG. 6 illustrates control parameters used when measuring the CI 50 using the measurement device 1. The control parameters include location parameters for the CPU 11 to identify the location of the discoloration area 30. The location parameters are L1, L2, and L3 described above. The control parameters also include a first color parameter that is used to determine an initial defect in the CI. The first color parameter is the color information of the discoloration area 30 before the sterilization process, i.e., the initial color of the discoloration area 30. By comparing the color value of the discoloration area 30 of the CI, measured before the sterilization process, with the color value indicated by the first color parameter in the control parameters of the CI, the CPU 11 determines whether or not there is a processing defect in the CI. Specifically, if a color difference between the measured color value of the discoloration area 30 of the CI before the sterilization process and the color value indicated by the first color parameter is at least a predetermined value, the CPU 11 determines that the CI has an initial defect.

The control parameters also include a second color parameter, which is used when determining the achievement degree of the sterilization process. The second color parameter is the color information of the discoloration area 30 after the sterilization process. By comparing the color value of the discoloration area 30 of the CI, measured after the sterilization process, with the color value indicated by the second color parameter in the control parameters of the CI, the CPU 11 determines whether the achievement degree of the sterilization process is acceptable or not. Specifically, if a color difference between the measured color value of the discoloration area 30 of the CI after the sterilization process and the color value indicated by the second color parameter is less than a predetermined value, the CPU 11 determines that the sterilization process is acceptable. Note that the configuration may be such that the achievement degree of the sterilization process is evaluated in three or more levels according to the color difference, instead of being acceptable or not. In addition, the control parameters of the CI include a conveyance parameter indicating a conveyance speed when conveying the CI into the measurement device 1, and a measurement parameter indicating a number of times the CI is measured by the spectrometer 200 and a time interval of the measurement.

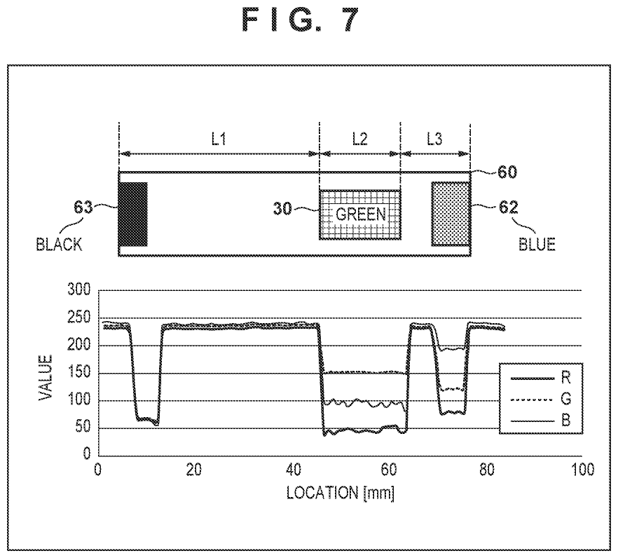

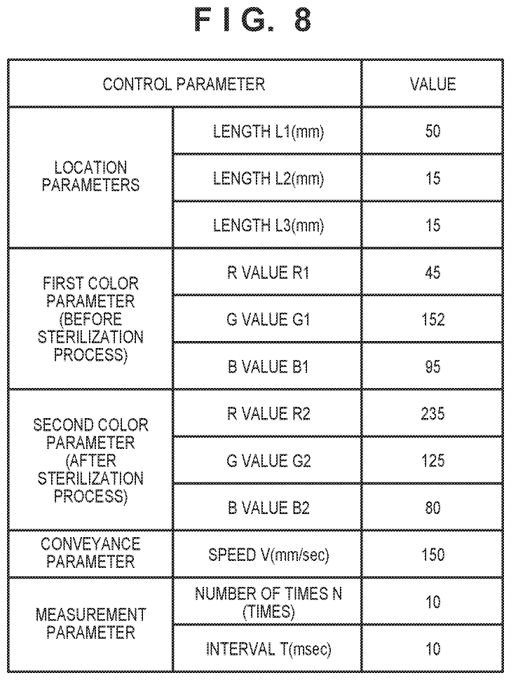

FIG. 7 illustrates another CI 60. The discoloration area 30 of the CI 60 is designed to be green before the sterilization process and change to orange after the sterilization process. The pattern 31 of the CI 60 has a black rectangle 63 on the first end side and a blue rectangle 62 on the second end side. The lower part of the CI 60 in FIG. 7 indicates the profile of the CI 60 before the sterilization process. FIG. 8 illustrates the control parameters of the CI 60.

The control parameters are provided corresponding to each type of CI that can serve as a measurement subject, and are stored in the non-volatile memory 12. When measuring a CI, the user inputs the type of CI to be measured, and whether the measurement is to be done before the sterilization process or after the sterilization process, into the control device 20. This information is communicated to the measurement device 1 from the control device 20. The measurement device 1 reads out, from the non-volatile memory 12, the control parameters corresponding to the type of CI which has been communicated, and performs measurement according to the control parameters that have been read out. Then, depending on whether the measurement has been taken before the sterilization process or after the sterilization process, the initial defect of the CI, the achievement degree of the sterilization process, or the like is determined according to the control parameters corresponding to the type of CI that has been communicated. Note that instead of storing the control parameters in the non-volatile memory 12 in advance, the control device 20 may be configured to communicate, to the measurement device 1, the control parameters corresponding to the type of the CI to be measured and whether the measurement is to be taken before the sterilization process or after the sterilization process each time a measurement is taken.

The configuration of the control device 20 in FIG. 3 will be described next. A storage unit 503 of the control device 20 stores the control parameters for each type of the CI. A processing unit 500 can add, update, and delete the control parameters stored in the storage unit 503. The processing unit 500 can display the control parameters stored in the storage unit 503 in a display unit 501. The user can also modify the displayed control parameters through an input unit 502. After the control parameters are modified by the user, the processing unit 500 can update the original control parameters in the storage unit 503 to the modified control parameters. Furthermore, the processing unit 500 can display a setting screen in the display unit 501 that allows the user to create control parameters for new types of CIs. The setting screen is a screen that allows the user to input the location parameters, the first color parameters, the second color parameters, the conveyance parameters, and the measurement parameters. Then, when the user inputs each parameter through the input unit 502, the control parameters of the new type of CI are stored (added) in the storage unit 503. Of the control parameters stored in the storage unit 503, the processing unit 500 can transmit control parameters selected by the user to the measurement device 1 over the communication line 15 and store those parameters in the non-volatile memory 12 of the measurement device 1. The processing unit 500 can also update or delete the control parameters stored in the non-volatile memory 12 of the measurement device 1. In other words, the non-volatile memory 12 is configured such that the control parameters can be added, updated, and deleted by the control device 20. As such, the control device 20 can be called an “information processing device”.

In this manner, the configuration is such that the control parameters of a CI can be added to the non-volatile memory 12 of the measurement device 1 through the control device 20, and the control parameters stored in the non-volatile memory 12 can be deleted or updated through the control device 20. This makes it possible to have the measurement device 1 measure various types of CIs. Furthermore, initial defects in various types of CIs, the achievement degree of sterilization processes performed using various types of CIs, and the like can be determined by the measurement device 1.

Pattern parameters, which indicate the color information, location information, and the like of the pattern 31, can also be added to the control parameters. The pattern parameters make it possible for the measurement device 1 to obtain the profile on the surface of the CI 4 and determine whether the CI 4 is being conveyed in the measurement device 1 with the first end or the second end serving as the leading end. Based on this determination result and the location parameters, the measurement device 1 can identify the discoloration area 30 and acquire the color information of the discoloration area 30 regardless of the orientation of the CI placed on the tray 2.

### Second Embodiment

A second embodiment will be described next, focusing on the differences from the first embodiment. In the first embodiment, the control parameters were created by the user using the control device 20 and transmitted to the measurement device 1. In the present embodiment, the location parameters in the control parameters can be set automatically.

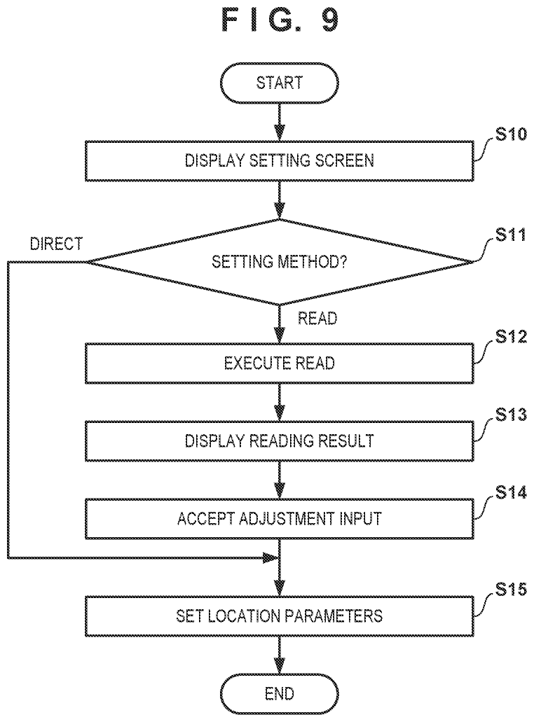

FIG. 9 is a flowchart illustrating processing performed by the determination system according to the present embodiment. In S10, the processing unit 500 of the control device 20 displays, to the user, a setting screen for setting the control parameters, in response to a user operation. In S11, the processing unit 500 accepts an input from the user pertaining to a method for setting the location parameters. If the user specifies direct input as the setting method, a screen for the user to input the location parameters is displayed and the location parameters are set in S15 based on the user input, as in the first embodiment.

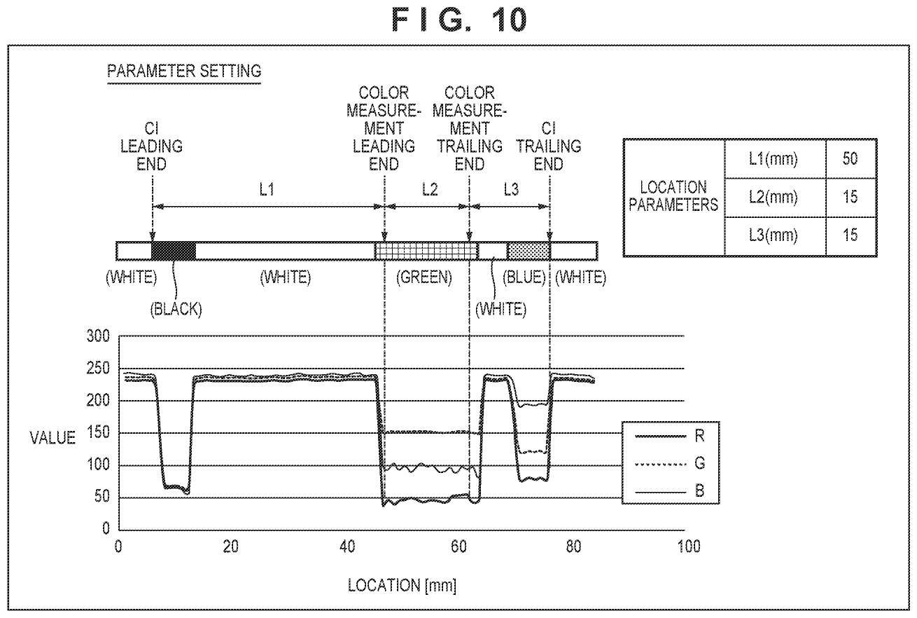

On the other hand, if reading input is specified in S11, the processing unit 500 displays a screen prompting the user to place the CI on the tray 2 of the measurement device 1. When the CI is placed on the tray 2, in S12, the measurement device 1 obtains the profile of the CI by reading the surface of the CI and transmits the obtained profile to the control device 20. The processing unit 500 determines the first end, the second end, the location of the first end side of the discoloration area 30, and the location of the second end side of the discoloration area 30 of the CI based on the profile of CI, and determines L1, L2, and L3 as a result. Then, in S13, the processing unit 500 displays the determined L1, L2 and L3 along with the profile as a reading result. FIG. 10 illustrates an example of the reading result displayed to the user. As illustrated in FIG. 10, the processing unit 500 can display the color of the surface of the CI based on the profile.

Note that the processing unit 500 determines the first end, the second end, the location of the first end side of the discoloration area 30, and the location of the second end side of the discoloration area 30 of the CI according to a predetermined criterion based on a change in the color of the CI surface. For example, the spectrometer 200 measures the white reference plate 9 before the CI reaches the measurement region, after the measurement region has exited, or the like. Accordingly, locations that have changed from white to other colors can be determined as the first end and the second end. Because the discoloration area 30 is long to a certain extent in the long side direction of the CI, a section having the same color in the long side direction for at least a predetermined length can be determined to be the discoloration area 30.

However, L1, L2, and L3 determined by the processing unit 500 may not be accurate, and thus in S14, the processing unit 500 accepts the input of adjustments from the user in response to the reading result. For example, the user can input, to the control device 20, which short side of the CI is the first end, as an adjustment input. The user can change the location of the first end, the location of the second end, the location of the end of the discoloration area 30 on the first end side, and the location of the discoloration area 30 on the second end side based on the result determined by the processing unit 500. After the user adjustment in S14 is complete, the processing unit 500 sets the location parameters in S15.

As described thus far, in the present embodiment, the measurement device 1 is caused to measure the surface of the CI, and the location parameters are set as a result. Accordingly, the amount of work the user must perform to create the control parameters can be reduced.

In the present embodiment, the first end and the second end are defined as the locations of the short sides of the CI. For example, in the CIs illustrated in FIGS. 5 and 7, the pattern 31 is at the first end or the second end of the CI, and thus in the CIs illustrated in FIGS. 5 and 7, the first end and the second end of the CI can be determined based on the pattern 31. On the other hand, in the CI illustrated in FIG. 4, the pattern 31 is not at the first end or the second end, and thus with the CI illustrated in FIG. 4, the end to be determined may be different from the first end or the second end of the CI. However, in such a case, it does not matter if the pattern 31 is located at the first end or the second end. This is because the location parameters are for identifying the location of the discoloration area 30, and the measurement device 1 can identify the location of the discoloration area 30 using the location of the pattern 31 as a reference. In other words, the configuration may be such that L1, L3, or the like is defined according to the distance between the discoloration area and the pattern 31, for example. Note that when there are two lines on the second end side, as in the CI illustrated in FIG. 4, the shortest distance between the discoloration area 30 and the pattern 31 may be L3, or the longest distance may be L3. The same applies to L1.

### Third Embodiment

A third embodiment will be described next, focusing on the differences from the first embodiment and the second embodiment. In the present embodiment, the first color parameter and the second color parameter can be set automatically.

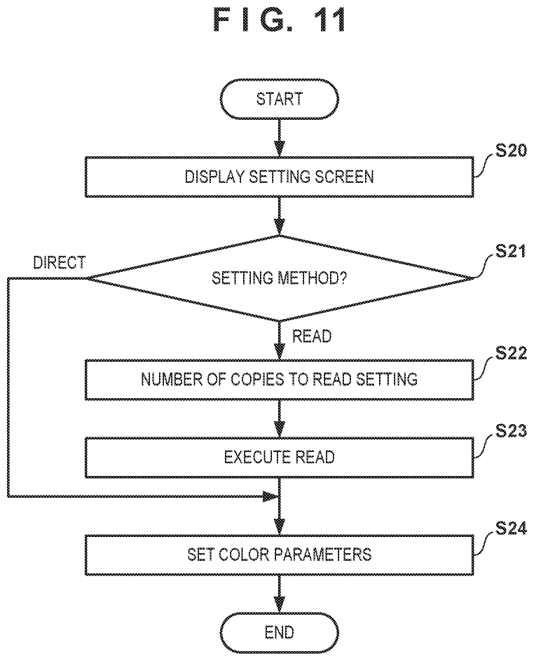

FIG. 11 is a flowchart illustrating processing performed by the determination system according to the present embodiment. Note that the first color parameter is color information of the discoloration area 30 before the sterilization process, the second color parameter is color information of the discoloration area 30 after the sterilization process, and the parameter set in the processing illustrated in FIG. 11 is the one of the first color parameter and the second color parameter specified by the user in S21. The following descriptions will refer to the first color parameter and the second color parameter collectively as “color parameters”. It is assumed that the location parameters of the CI for which the color parameters are to be set is set in the control device 20 through direct input or reading input as described in the second embodiment before executing the processing illustrated in FIG. 11.

In S20, the processing unit 500 displays, to the user, a setting screen for setting the control parameters, in response to a user operation. In S21, the processing unit 500 accepts an input from the user pertaining to a method for setting the color parameters. If the user specifies direct input as the setting method, a screen for the user to input the color parameters is displayed and the color parameters are set in S24 based on the user input, as in the first embodiment.

On the other hand, if reading input is specified in S21, in S22, the processing unit 500 accepts user input pertaining to the number of CIs to be read. Then, in S23, the control device 20 causes the measurement device 1 to read the colors in the discoloration area 30 in order. Because the location parameters are already set, the measurement device 1 can determine the location of the discoloration area on the CI. Once the measurement device 1 reads the specified number of CIs, the processing unit 500 obtains the reading results from the measurement device 1 and calculates the color parameters. The color parameters can be found by averaging the color information of the discoloration area 30 of the CIs which have been read. The processing unit 500 sets the color parameters calculated in S24.

As described thus far, in the present embodiment, the measurement device 1 is caused to measure the discoloration area 30 of the CI, and the color parameters are set as a result. Accordingly, the amount of work the user must perform to create the control parameters can be reduced.

### Fourth Embodiment

Next, a fourth embodiment will be described, focusing on the differences from the first embodiment to the third embodiment. In the present embodiment, the control parameters are obtained over a network such as the Internet. It is assumed that the CI control parameters are stored in a server device on a network operated by the manufacturer, distributor, or the like of the CI, and are available to the public. The processing unit 500 is configured to be capable of, for example, accessing a server device on the Internet via a LAN and obtaining control parameters published by the server device.

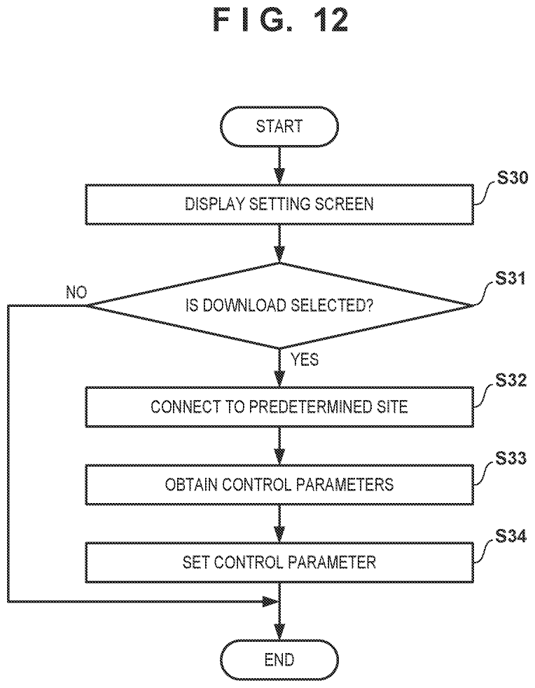

FIG. 12 is a flowchart illustrating processing performed by the determination system according to the present embodiment. In S30, the processing unit 500 displays, to the user, a setting screen for setting the control parameters, in response to a user operation. In S31, the processing unit 500 accepts an input from the user pertaining to a method for obtaining the control parameters. When the user selects “download” as the obtainment method, in S32, the processing unit 500 accesses a server device on a network according to the CI, and obtains the control parameters in S33. Then, in S34, the processing unit 500 sets the control parameters. If “download” is not selected as the method for obtaining the control parameters in S31, the processing unit 500 ends the processing of FIG. 12. In this case, the user sets the control parameters as described in the first embodiment, the second embodiment, or the like, for example.

As described thus far, in the present embodiment, the control parameters are obtained from a server device on a network, and thus it is not necessary for the user to create the control parameters, which makes it possible to reduce the amount of work the user must perform.

### OTHER EMBODIMENTS

In the foregoing embodiments, the first color parameter and the second color parameter are stored in the measurement device 1 as control parameters for the measurement device 1 to determine initial defects, the achievement degree of the sterilization process, and the like. However, a configuration can also be employed in which the control device 20 determines initial defects, the achievement degree of the sterilization process, and the like. In this case, the measurement device 1 measures the color of the discoloration area 30 and transmits color information indicating the measured color to the control device 20, and the control device 20 then determines initial defects, the achievement degree of the sterilization process, and the like based on the first color parameter, the second color parameter, and the like. As such, in this case, the first color parameter and the second color parameter need not be stored in the non-volatile memory 12 of the measurement device 1. However, the control device 20 requires the first color parameter and the second color parameter. In other words, in the determination system of the present invention, there are cases where the control parameters are stored in a distributed manner in both the control device 20 and the measurement device 1, instead of being stored only in the measurement device 1.

Additionally, although the descriptions of the foregoing embodiments were based on a determination system that determines the achievement degree of a sterilization process, the present invention is not limited to a determination system that determines the achievement degree of a sterilization process. For example, an indicator similar to a CI is used in medical instrument cleaning processes as well. As such, the present invention can also be applied to a determination system that determines the achievement degree of a cleaning process. More generally, the present invention can be applied to any process performed using an indicator (test paper) having a discoloration area that changes color in accordance with the achievement degree of the process.

Embodiments of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiments and/or that includes one or more circuits (e.g., application specific integrated circuit (ASIC)) for performing the functions of one or more of the above-described embodiments, and by a method performed by the computer of the system or apparatus by, for example, reading out and executing the computer executable instructions from the storage medium to perform the functions of one or more of the above-described embodiments and/or controlling the one or more circuits to perform the functions of one or more of the above-described embodiments. The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the present invention, various types of indicators can be measured.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A determination system comprising:
a measurement unit configured to measure color information on a surface of an indicator having a discoloration area in which a color changes according to an achievement degree of a predetermined process;
a determination unit configured to determine an initial defect in the indicator or the achievement degree of the predetermined process based on the color information of the discoloration area measured by the measurement unit;
a storage unit configured to store a control parameter corresponding to a type of the indicator; and
a processing unit configured to perform processing of setting the control parameter in the storage unit,
wherein the measurement unit is further configured to measure the color information of the discoloration area of the indicator based on the control parameter, stored in the storage unit, that corresponds to the type of the indicator to be measured.

2. The determination system according to claim 1, wherein the control parameter corresponding to the type of the indicator includes a location parameter for specifying a location of the discoloration area in an indicator of the type.

3. The determination system according to claim 2, further comprising:
an input unit configured to enable a user to input the location parameter.

4. The determination system according to claim 3, wherein the processing unit is further configured to perform processing of determining and displaying the location parameter based on the color information of the surface of the indicator measured by the measurement unit, and
the input unit is further configured to enable the user to input the location parameter by enabling the user to adjust the location parameter determined and displayed by the processing unit.

5. The determination system according to claim 2, wherein the determination unit is further configured to determine the initial defect of the indicator or the achievement degree of the predetermined process based on the control parameter, stored in the storage unit, that corresponds to the type of the indicator to be measured.

6. The determination system according to claim 5, wherein the control parameter corresponding to the type of the indicator includes a first color parameter that specifies an initial color of the discoloration area of the indicator of the type, and
the determination unit is further configured to determine the initial defect of the indicator by comparing the color information of the discoloration area measured by the measurement unit with color information specified by the first color parameter of the control parameter.

7. The determination system according to claim 6, further comprising:
an input unit configured to enable a user to input the first color parameter.

8. The determination system according to claim 6, wherein the processing unit is further configured to perform processing of obtaining the first color parameter by causing the measurement unit to measure the color information of the discoloration area of the indicator based on the location parameter.

9. The determination system according to claim 5, wherein the control parameter corresponding to the type of the indicator includes a second color parameter for specifying a color of the discoloration area in an indicator of the type after the predetermined process, and
the determination unit is further configured to determine the achievement degree of the predetermined process by comparing the color information of the discoloration area measured by the measurement unit with color information specified by the second color parameter of the control parameter.

10. The determination system according to claim 9, further comprising:
an input unit configured to enable a user to input the second color parameter.

11. The determination system according to claim 10, wherein the processing unit is further configured to perform processing of obtaining the second color parameter by causing the measurement unit to measure the color information of the discoloration area of the indicator based on the location parameter.

12. The determination system according to claim 1, wherein the processing unit is further configured to add the control parameter to the storage unit, update the control parameter stored in the storage unit, or delete the control parameter stored in the storage unit.

13. The determination system according to claim 12, wherein the processing unit is further configured to be capable of obtaining the control parameter over a network.

14. The determination system according to claim 1, wherein the determination system comprises:
a measurement device including the measurement unit, the determination unit, and the storage unit; and
a control device including the processing unit.

15. The determination system according to claim 1, wherein the determination system comprises:
a measurement device including the measurement unit; and
a control device including the determination unit and the processing unit,
wherein the storage unit is provided in each of the measurement device and the control device.

16. A measurement device in a determination system, the determination system determining an achievement degree of a predetermined process based on a color of a discoloration area of an indicator in which a color changes according to the achievement degree of the predetermined process, the measurement device comprising:
a measurement unit configured to measure color information on a surface of the indicator; and
a storage unit configured to store a control parameter corresponding to a type of the indicator,
wherein the measurement unit is further configured to measure the color information of the discoloration area of the indicator based on the control parameter that corresponds to the type of the indicator to be measured, and
the storage unit is further configured to be capable of adding, updating, or deleting the control parameter from another device of the determination system.

17. The measurement device according to claim 16, wherein the control parameter corresponding to the type of the indicator includes a location parameter for specifying a location of the discoloration area in an indicator of the type.

18. An information processing device capable of communicating with a measurement device, the measurement device including a measurement unit for measuring color information on a surface of an indicator and determining an achievement degree of a predetermined process based on the color information, the information processing device comprising:
a transmitting unit configured to transmit a control parameter to the measurement device; and
a storage unit configured to store a control parameter corresponding to a type of the indicator,
wherein the transmitting unit is further configured to transmit a control parameter corresponding to a type of the indicator to be measured, and
the storage unit is further configured to be capable of adding, updating, or deleting the control parameter.
